(19) United States
(12) Patent Application Publication
(10) Pub. No.: US 2020/0095421 A1
(43) Pub. Date: Mar. 26, 2020

(54) Water-Containing Hydrogel Composition Comprising Elemental Silver Particles

(71) Applicant: Paul Hartmann AG, Heidenheim (DE)

(72) Inventor: Markus KETTEL, Heidenheim (DE)

(73) Assignee: Paul Hartmann AG, Heidenheim (DE)

(21) Appl. No.: 16/472,149

(22) PCT Filed: Dec. 22, 2017
(86) PCT No.: PCT/EP2017/084410

§ 371 Date: Jun. 20, 2019

(30) Foreign Application Priority Data
Dec. 23, 2016 (DE) 10 2016 125 579.3

(51) Int. Cl. C08L 75/04; A61L 26/00; C08K 3/08; A61L 15/26; C08G 18/32

(52) U.S. Cl. CPC C08L 75/04; A61L 26/008; A61L 26/0019; C08K 3/08; C08K 5/053; C08G 18/3225; C08K 2003/0806; C08K 2201/003; C08K 2201/005; A61L 15/26

(57) ABSTRACT

The invention relates to a water-containing hydrogel composition comprising elemental silver particles and to a multi-layered wound dressing comprising the hydrogel composition. The multi-layered wound dressing is used especially in the inflammation and/or granulation phases in the treatment of, for example, burns and/or chronic wounds. In addition, the invention relates to a process for preparing the hydrogel composition.

The invention relates to a water-containing hydrogel composition comprising elemental silver particles and to a multi-layered wound dressing comprising the hydrogel composition. The multi-layered wound dressing is used especially in the inflammation and/or granulation phases in the treatment of, for example, burns and/or chronic wounds. In addition, the invention relates to a process for preparing the hydrogel composition.

### BACKGROUND OF THE INVENTION

In the literature, a wound is understood to mean the severance of the cohesion of tissues in the body shell in human beings or animals. It may be accompanied by a loss of substance. The healing of skin wounds is based on the ability of the skin to regenerate epithelial, connective and supporting tissue. The regeneration itself is characterised by a complex interplay of overlapping cellular activities which advance the healing process step by step. Three main wound healing phases are described in the literature, irrespective of the nature of the wound. These are the inflammatory or exudative phase for haemostasis and wound cleansing (phase 1, inflammation phase), the proliferative phase for building up granulation tissue (phase 2, granulation phase) and the differentiation phase for epithelialisation and scar formation (phase 3, epithelialisation phase).

In order to support the different wound healing phases, the use of wound dressings is of great importance. In WO 2010/000451 for example, a multi-layered wound dressing with a hydrogel matrix, and in WO 2011/141454 a wound dressing for treating wounds with a super-absorbent material are described, which are each especially suitable for keeping the wound in a moist environment.

WO 2010/000451 describes in particular hydrogels for the hydrotherapy of wounds.

In order to prevent and treat wound infections, wound dressings are important in the state of the art which additionally exhibit antimicrobial properties.

Elemental silver has been successfully used for many years as an antimicrobial agent in the treatment of wounds. Because of the electrochemical potential of elemental silver, only small numbers of silver ions are released, which are effective in inhibiting or killing bacteria. One particular advantage of silver ions is that no or hardly any resistances have been found in bacteria so far. It should be noted in this connection that while increasing the number of silver ions released is beneficial for the antimicrobial activity, it also increases the risk of an undesirable absorption of silver in the human organism. Absorbed silver cannot be eliminated by the body, but is deposited in the tissue in the form of elemental silver, which in extreme cases can lead to irreversible discoloration of the skin (argyrosis or argyria).

### Problem and Brief Description of the Invention

One problem of the present invention was to find an antimicrobial composition containing silver which enabled a sufficiently high silver ion release to ensure effective antimicrobial activity during the treatment of wounds, while at the same time successfully preventing the release of silver particles and their absorption in the human body.

The present invention is intended to provide a system for wound therapy with which the treatment of wounds can be performed as effectively as possible and which exhibits high antimicrobial activity. In addition, it is intended to promote and accelerate wound healing. The intention is also to provide a wound dressing which exhibits great wearing comfort and exhibits effective antimicrobial efficacy even when worn for a long time.

The problem has surprisingly been solved by the provision of a hydrogel composition comprising elemental silver particles, a multi-layered wound dressing comprising the hydrogel composition and a process for preparing the hydrogel composition.

One subject matter of the invention is therefore a water-containing hydrogel composition comprising elemental silver particles with an average particle diameter, determined by transmission electron microscopy (TEM), of 5-20 nm, and a particle size distribution, determined by laser diffractometry, of D90≤25 nm, the silver content, based on the total weight of the hydrogel composition, being between 15 and 500 ppm.

A further subject matter of the invention consists in the provision of a multi-layered wound dressing comprising at least one water-impermeable and water vapour-impermeable support layer, an absorbent layer and a layer comprising the water-containing hydrogel composition according to the present invention.

A further subject matter of the present invention is the provision of a process for preparing a hydrogel composition, comprising reacting a mixture containing:
(a) a polyamine,
(b) optionally a non-ionic surfactant,
(c) further optionally a polyhydric alcohol selected from propylene glycol and/or glycerol,
(d) elemental silver particles having an average particle diameter, determined by transmission electron microscopy (TEM), of 5-20 nm and a particle size distribution, determined by laser diffractometry, of D90≤25 nm, and
(e) water,

with an aliphatic diisocyanate polymer in order to form a water-containing hydrogel composition in which the silver content, based on the total weight of the hydrogel composition, is between 15 and 500 ppm.

### DETAILED DESCRIPTION OF THE INVENTION

It has been surprisingly shown in the present invention that a strong antimicrobial effect can be achieved if the silver particles are contained in a hydrogel and the hydrogel has a silver content of between 15 and 500 ppm (or 0.0015 to 0.05% w/w), the silver particles having an average particle diameter of 5-20 nm.

The term “hydrogel composition” will also be referred to synonymously below as “hydrogel” or “hydrogel matrix”.

In the hydrogel composition of the present invention, the benefits of a hydroactive wound dressing that can absorb wound exudate and does not have any traumatic effects on the wound are combined with the antimicrobial properties of silver nanoparticles. In particular, elemental nanosilver is incorporated homogeneously into the structure of a hydrogel, which is usually cross-linked.

The term “nanosilver” is used for elemental silver particles with an average particle diameter of 5-20 nm, preferably 9-18 nm. The particle diameter can be determined by transmission electron microscopy (TEM) for example.

In particular, the silver particles have a narrow numerical particle size distribution of D90≤25 nm, preferably D90≤20 nm, particularly preferably D90≤18 nm. In particular, the particle size distribution is D99≤25 nm, preferably D99≤20 nm, particularly preferably D99≤18 nm. The D90 (or D99) value indicates that 90% (or 99% respectively) of the particles are below the size stated. The particle size distribution can be determined by laser diffractometry for example.

Elemental silver particles of this kind are commercially available as Agpure® W10 (RAS materials GmbH, Regensburg, Germany) in the form of an aqueous nanosilver dispersion with an amount of 10% by weight (w/w) silver. The dispersion additionally contains <10% by weight Tween 20 and polysorbitol as stabilising agents.

The silver content of the hydrogel composition, based on the total weight of the hydrogel composition, is usually between 15 and 500 ppm, preferably between 25 and 250 ppm, particularly preferably between 75 and 200 ppm.

The antimicrobial efficacy of the silver-containing hydrogel composition of the invention is enabled by the controlled release of silver ions from the hydrogel caused by oxidation and ion transport through the aqueous system of the hydrogel. In the process, there may be a controlled oxidation of the elemental nanosilver particles via oxygen present or transported in the aqueous phase to the elemental silver nanoparticles. The controlled silver ion transport is achieved in particular by the aqueous phase of the hydrogel in the aqueous or exudative environment. In this way, a controllable release of silver ions is achieved, which enables great antimicrobial efficacy even when there is only a low nanosilver concentration in the hydrogel.

It is preferable that at least 3 log levels of germ reduction are achieved, as can be determined by a zone of inhibition assay for example.

Thanks to the low release of silver particles, the hydrogel compositions of the present invention are not cytotoxic to human cells and do not usually cause any allergic reactions.

The water content of the hydrogel composition of the invention is preferably 20 to 90% by weight, especially 30 to 85% by weight, in particular 40 to 80% by weight and very particularly preferably 50 to 75% by weight water. In this way, it is possible to provide a wound dressing that provides a sufficient amount of moisture for natural wound healing.

As water-containing hydrogel compositions in connection with the present invention, it is possible in particular to use compositions which form a coherent, discrete layer and which do not release any water under pressure.

The hydrogel composition can preferably comprise a hydrophilic polyurethane foam. As the polyurethane foam in connection with the present invention, it is possible to use any hydrophilic polyurethane foam that is conventionally used in modern wound treatment today and which can take up an amount of water into its polyurethane matrix and possesses sufficient absorption. This means that in connection with the present invention, a hydrophilic polyurethane foam is to be understood as meaning a polyurethane foam which can take up and store, i.e. absorb, liquid in its polyurethane matrix and pores and can release at least part of the absorbed liquid again. In this context, open-pored, hydrophilic polyurethane foams in particular are suitable as hydrophilic polymer foams.

Alternatively or in addition, the hydrogel may also comprise a super-absorbent material, preferably an anionic, super-absorbent material, usually a polymer, especially an at least partially cross-linked polymer, for example polyacrylate-based.

In particular, hydrogel compositions are suitable in connection with the present invention which comprise a polyurethane-polyurea copolymer. That polyurethane-polyurea copolymer may in particular be formed from a prepolymer with aliphatic diisocyanate groups and a polyamine based on polyethylene oxide. In particular, the polyurethane-polyurea copolymer may be formed from a prepolymer with isophorone diisocyanate ends, a polyamine based on polyethylene oxide, and water. These hydrogel compositions are particularly suitable for storing water and releasing that water to a wound.

It is also preferable for the water-containing hydrogel composition also to comprise at least one polyhydric alcohol from the group of dihydric, trihydric, tetrahydric, pentahydric or hexahydric alcohols. In particular, the alcohol can be selected from the group of glycols, especially ethylene glycol or propylene glycol, and sorbitol or glycerol, or mixtures thereof. These alcohols are excellent moisturisers and thus constitute a nourishing component for the skin surrounding the wound.

The water-containing hydrogel composition can in particular comprise 0 to 50% by weight of a polyhydric alcohol. In particular, the hydrogel composition comprises 5 to 40% by weight of a polyhydric alcohol and very particularly preferably 10 to 30% by weight of a polyhydric alcohol.

It is particularly preferable for the hydrogel composition to contain a polyhydric alcohol selected from propylene glycol and/or glycerol, preferably glycerol. The amount of propylene glycol and/or glycerol in the hydrogel composition is in particular 5 to 30% by weight, based on the total weight of the hydrogel composition, preferably 10 to 25% by weight, particularly preferably 15 to 20% by weight.

All together, it may be contemplated according to the present invention that the water-containing hydrogel composition comprises at least 10% by weight, preferably at least 15% by weight polyurethane-polyurea copolymer. In this context, it may further preferably be contemplated that the hydrogel composition is formed from 6 to 60% by weight of a prepolymer with aliphatic diisocyanate groups, 4 to 40% by weight polyethylene oxide-based polyamine, 0 to 50% by weight of a polyhydric alcohol and at least 20% by weight water.

It may further preferably be contemplated that the hydrogel composition is formed from 6 to 30% by weight of a prepolymer with aliphatic diisocyanate groups, 4 to 20% by weight polyethylene oxide-based diamine, 10 to 30% by weight of a polyhydric alcohol selected from the group consisting of propylene glycol and/or glycerol and at least 30% by weight water.

It may very particularly preferably be contemplated that the hydrogel composition is formed from 6 to 20% by weight of a prepolymer with isophorone diisocyanate ends, 4 to 15% by weight polyethylene oxide-based diamine, 15 to 20% by weight polypropylene glycol and/or glycerol and at least 20% by weight water.

This hydrogel composition usually has a free absorption A3 (measured according to DIN EN 13723-1 (2002)) of at least 1 g/g and no more than 5 g/g, provides a non-irritating, liquid-absorbing, padding and skin-like medium that protects against bacteria, and is thus particularly good as a wound contact layer.

Wound dressings comprising a hydrogel matrix the layer thickness of which is 0.1 to 5.0 mm have proven particularly advantageous embodiments. In particular, a wound dressing of the invention therefore has a wound contact layer with a layer thickness of 0.1 to 5.0 mm, especially 0.5 to 5.0 mm and most particularly preferably 0.5 to 3.0 mm. Wound dressings with layer thicknesses like this do not exhibit any wound adhesion on the one hand, and are capable, on the other hand, of taking up any wound exudate oozing from a wound and passing it on to the absorbent layer. These layer thicknesses can be the same at every part of the wound contact layer or may vary in different regions of the wound contact layer.

In addition, it is preferable for the hydrogel matrix to comprise channels, especially conical channels, to enable liquids to pass from the first to the second side. In this way it is in particular possible to provide a better passage for wound exudate. It is particularly preferably contemplated that the channels should have an elliptical or circular cross-section, i.e. that the channels have a circular or elliptical aperture both on the first and on the second side of the hydrogel matrix, the circular or elliptical apertures on the first and second sides differing in size. It may, however, also be contemplated that the channels have a triangular, rectangular, square, pentagonal, hexagonal or some other polygonal cross-section. It is very particularly preferably contemplated in this connection that the first side should have apertures which are larger than the apertures on the second side.

In another preferred embodiment, the hydrogel composition consists 10 to 25% by weight, preferably 15 to 20% by weight of a solid polymeric portion and 75 to 90% by weight, preferably 80 to 85% by weight of a liquid portion. The amount of purified water in the liquid portion is usually in a range of 40 to 90% by weight, preferably 50 to 80% by weight and the amount of glycerol is in a range of 5 to 40% by weight, preferably 10 to 30% by weight.

The polymeric portion has, for example, a weight ratio of an NCO-terminated 3-armed poly(ethylene oxide-stat-propylene oxide) (ratio PEO/PPO: 70:30 or 80:20) to an amine-terminated linear poly(ethylene oxide co-propylene oxide) of 1.0 to 2.0, preferably 1.2 to 1.8.

A typical prepolymer with aliphatic diisocyanate groups is commercially available as, for example, Aquapol® PL-13000-3 (Carpenter; Richmond, USA), which contains 4-7% by weight isophorone diisocyanates.

A typical polyamine is commercially available as, for example, Jeffamin® ED-2300, Huntsman; Everberg, Belgium, which contains a water-soluble aliphatic polyether amine derived from propylene oxide-terminated polyethylene glycol.

The hydrogel composition is in particular free of chloride ions, as contained, for example, in some standard commercial hydrogels in the form of isotonic saline solutions, in order to prevent the silver particles from precipitating in the form of silver chloride, which would reduce their efficacy.

In addition, the hydrogel composition can comprise a non-ionic surfactant. This is intended in particular to achieve stabilisation and a homogeneous distribution of the silver particles in the hydrogel composition. In particular, hydrophobic stabilisation and binding of the silver particles in the polymer structure of the hydrogel can be achieved by means of stabilisers, e.g. by the use of surfactants such as Tween 20, polyethylene glycol, sorbitol, polyvinyl pyrrolidone and mixtures thereof, especially by Tween 20 or sorbitol.

In one particularly advantageous embodiment, the water-containing hydrogel composition comprises 6 to 20% by weight of a prepolymer with isophorone diisocyanate ends, 4 to 15% by weight of a polyethylene oxide-based diamine, 15 to 20% by weight polypropylene glycol and/or glycerol, 40 to 70% by weight water and 25 to 250 ppm elemental silver with an average particle diameter of 9-18 nm, all weight statements being based on the total weight of the hydrogel composition.

In a further aspect, the present invention relates to a process for preparing a hydrogel composition as described above.

The process preferably comprises reacting a mixture containing:
(a) a polyamine,
(b) optionally a non-ionic surfactant,
(c) further optionally a polyhydric alcohol selected from propylene glycol and/or glycerol,
(d) elemental silver particles with an average particle diameter of 5-20 nm, determined by transmission electron microscopy (TEM), and a particle size distribution, determined by laser diffractometry, of D90≤25 nm, and
(e) water,

with an aliphatic diisocyanate polymer in order to form a water-containing hydrogel composition in which the silver content, based on the total weight of the hydrogel composition, is between 15 and 500 ppm.

With regard to preferred embodiments of components (a) to (e), reference is made to the explanations on the composition of the hydrogel of the invention, which apply equally to the process of the invention.

In a further aspect, the invention relates to a multi-layered wound dressing comprising at least one water-impermeable and water vapour-impermeable support layer, an absorbent layer and a layer comprising the water-containing hydrogel composition.

Polymer films or polymer foams in particular can be used as the support layer, preferably films or foams produced from polyurethane, polyether urethane, polyester urethane, polyether-polyamide copolymers, polyacrylate or polymethacrylate. In particular, a water-impermeable and water vapour-impermeable polyurethane film or a water-impermeable and water vapour-impermeable polyurethane foam is suitable as the support layer. In particular, a polyurethane film, polyester urethane film or polyether urethane film is preferable as a polymer film. Most particularly, however, those polymer films are preferable which have a thickness of 15 μm to 50 μm, especially 20 μm to 40 μm and very particularly preferably 25 μm to 30 μm. The water vapour impermeability of the polymer film of the wound dressing is preferably at least 750 g/m²/24 h, especially at least 1,000 g/m²/24 h and very particularly preferably at least 2,000 g/m²/24 h (measured according to DIN EN 13726). In particularly preferred embodiments, these films have a moisture-tight, adhesive edge portion. This edge portion ensures that the wound system can be applied and fixed at the location of its intended use. Furthermore, it ensures that no liquid can escape between the film and the skin surrounding the area to be treated. It has been found that those adhesives are particularly preferable which, when applied in a thin layer of 20 g/m² to 35 g/m², together with the film, achieve a water vapour impermeability of at least 800 g/m²/24 h and preferably at least 1,000 g/m²/24 h (measured according to DIN EN 13726).

In a preferred embodiment, the absorbent layer comprises a hydrophilic polyurethane foam and the hydrogel. The surface of a hydrophilic polyurethane foam may, however, be impregnated or coated with the hydrogel or completely or partially permeated with it.

In an alternative embodiment, the hydrogel composition may also be present adjacent to or spatially separated from the absorbent layer. The hydrogel composition, for example comprising a polyurethane-polyurea copolymer, may be coated on a surface of a layer of a polyurethane foam, for instance, so that a hydrogel layer comprising the hydrogel composition rests in direct contact on a layer of polyurethane foam. Alternatively, the hydrogel layer and the absorbent layer can also be separated from one another by a spacing layer. The spacing layer can, for example, comprise a hydrogel matrix, a polymer film, a hydrocolloid matrix, a polymer network, a fabric, an adhesive and/or a polymer network.

In addition, the multi-layered wound dressing can also comprise further layers apart from the absorbent layer and the support layer, such as a wound contact layer, one or more barrier layers and/or one or more distribution layers.

Preferred wound dressings comprise a support layer, a hydrogel layer in accordance with the present invention and optionally an absorbent layer disposed between the hydrogel layer and the support layer. The absorbent layer can preferably comprise a fibrous material, particularly preferably a hydrophilic polyurethane foam. The hydrogel layer may be continuous or discontinuous. It may, for example, be applied over the whole area of the support layer or may have channels, holes or apertures shaped in different ways. In the case of a discontinuous hydrogel layer, a plurality of discrete hydrogel elements may be applied to the support layer and/or the absorbent layer, which may be in the shape of circles, squares or other regular or irregular polygons.

Possible arrangements of the various layers in multi-layered wound dressings of the invention are described in WO 2010/000450, for example, which is hereby incorporated in full by reference.

The wound dressings are also very comfortable for the patient, since they are easy to use, are skin-friendly, soft and thin, adapt to the skin and have an analgesic effect (because of a hydrogel cooling effect) and can thus be used over a long period, usually 3 to 5 days, before the wound dressing is changed.

As described at the beginning, the healing process can typically be divided into different phases, irrespective of the nature of the wound. A distinction is made in the art between the inflammation (cleaning phase), granulation (proliferation) and epithelialisation phases. A common feature is that different cell types interact with one another and are activated and proliferate.

The risk of wound infection is greatest in the inflammation phase and granulation phase, since it is then that bacterial germs can enter the wound most easily. The advantageous effects achieved by the wound dressing of the invention are thus particularly pronounced in these phases.

The antibacterial effect is especially important in the case of slow-healing wounds, such as burns or chronic wounds. The wound dressing of the invention is therefore particularly suitable for treating burns and/or chronic wounds, especially wounds of the kind that occur in cases of decubitus ulcers, venous ulcers and diabetic syndrome.

### DESCRIPTION OF THE FIGURES

FIG. 1: Release of silver nanoparticles from the hydrogel into the aqueous medium

FIG. 2: Release of silver ions from the hydrogel into the aqueous medium

FIG. 3A/B: Antimicrobial activity of the hydrogel at different silver contents

FIG. 4: Cytotoxicity and cell compatibility of the hydrogel at different silver contents

FIG. 5A/B: Absorption capacity of a hydrogel of the invention at different silver contents

FIG. 6A/B: Dehydration of a hydrogel of the invention at different silver contents

### EXAMPLES

Preparation of a Hydrogel Composition

In a first step, a mixture of 52.5% by weight polyamine (Jeffamin® ED-2003, Huntsman; Everberg, Belgium) and 47.5% by weight water is mixed. Of that mixture, 132.5 g are mixed with 200 g glycerol, 567.5 g water and the corresponding amount of an aqueous silver nanoparticle suspension (Agpure® W10, RAS materials GmbH, Regensburg, Germany) with a nominal silver content of 10% by weight, a particle size distribution of D99<20 nm and an average particle size of 15 nm. 100 g of an IPDI-based prepolymer (Aquapol® PI-13000-3; Carpenter; Richmond, USA) are added to that mixture. The components are thoroughly blended, the still liquid gel is portioned into petri dishes and polymerises completely there.

Jeffamin Mix:

| | | |
|---|---|---|
| Jeffamin ED-2003 | Huntsman; Everberg, Belgium | 52.5% by weight |
| Aqua purificata | water treatment plant | 47.5% by weight |

Aquapol:

| | | |
|---|---|---|
| Aquapol PI-13000-3 | Carpenter; Richmond, USA | 100.0% by weight |

Components for 1 kg hydrogel:

| Chemical | Amount weighed in [g] | Proportion of gel [%] |
|---|---|---|
| glycerol | 200 | 20 |
| water | 567.5 | 56.75 |
| Jeffamin mix | 132.5 | 13.25 |
| Aquapol | 100 | 10 |
| silver | 0.0125 to 0.25 | 0.00125 to 0.025 (12.5 ppm |
| | | to 250 ppm) |

Measuring Methods

Nanoparticle Release

Production of the Calibration Curve

In order to investigate the migration of the silver nanoparticles from the gel into the aqueous medium, the eluate of the ion release is analysed by means of a photometer. The nanoparticles have an absorption peak in the visible range at approx. 410 nm. In order to be able to determine the concentration of the silver nanoparticles in the eluate, standards in concentrations of 50; 25; 10; 1; 0.1 mg Ag Pure® W 10/kg demineralised water are measured, with which a calibration curve can be produced.

Determining the Silver Nanoparticles Released

In order to determine the ion release, test pieces with a diameter of 50 mm are punched out, removed from the petri dish and weighed. The gels are transferred to 100 mL Erlenmeyer flasks, 30 mL water (HPLG grade) is added, and sealed with a ground-glass stopper. After that the samples are incubated at room temperature on a shaker for 24 hours at 130 rpm. After incubation for 24 hours, the extinction of the eluate at 410 nm is measured in the photometer. Polystyrene cuvettes are used.

Ion Release

In order to determine the ion release, test pieces with a diameter of 50 mm are punched out, removed from the petri dish and weighed. The gels are transferred to 100 mL Erlenmeyer flasks, 30 mL water (HPLG grade) is added, and sealed with a ground-glass stopper. After that the samples are incubated at room temperature on a shaker for 24 hours at 130 rpm. Following that, approx. 5-6 mL of the eluates are pipetted into brown vials and mixed with 20 μL concentrated nitric acid for stabilisation purposes. The samples are examined by IPC-MS in accordance with EN ISO 17294-2 (E29).

Release Kinetics

In order to observe the release of the silver nanoparticles or silver ions over a longer period, the corresponding measurements are determined at assay times of 2; 4; 6; 8; 24; 48 and 72 hours. Test pieces with a diameter of 50 mm are punched out of the AgNP gels, drawn from the petri dish and weighed. After that, the gels are treated as described in the corresponding sections.

Efficacy Studies

Working Cultures and Media

For the zone of inhibition and soft agar method, 24-hour cultures of the bacterial strain Staphylococcus aureus DSM 346 are used. For this purpose, a cryosphere is transferred to 9 mL caso broth and incubated in the incubator at 37° C. for 24 hours. The suspension is visually inspected for high turbidity and hence good growth.

Pour Plate Method

The viable bacteria are evaluated by counting the colony forming units (CFU). A defined quantity of bacterial suspension is mixed in warm caso agar and counted after incubation at the appropriate temperature. It is presumed that each bacterial cell forms a colony on the plate.

In the pour plate method, appropriate dilution levels of the samples in steps of 10 (1:10) are prepared and 1 mL thereof in each case is poured into an empty petri dish. After that approx. 20 mL are poured onto caso agar in the petri dish heated to 45° C. and the plate is swung in a figure-of-8-shaped movement so that the bacterial cells are distributed evenly in the agar. The plates are stored at room temperature until they have solidified. Finally, they are incubated in the incubator for approx. 24 hours at 37° C. and the bacteria colonies formed are counted. For evaluation purposes, the CFU/mL are calculated.

CFU: colonies counted

DF soft agar: dilution factor soft agar

DF D/e neutraliser: dilution factor soft agar

DF plate pour: dilution factor of the plates counted

CFU/mL=counted CFU*DF soft agar*DF D/e−neutraliser*DF plate pour

Zone of Inhibition Assay

In order to obtain a first impression of the antimicrobial efficacy of the AgNP gels, the samples are placed on freshly inoculated agar plates and assessed as to whether the samples inhibit the growth of the bacteria. For the zone of inhibition method, circular test pieces with a diameter of 30 mm are punched out. 5 mL of a bacteria suspension, 1 mL suspended in 150 mL liquid caso agar, are pipetted onto finished caso plates, and then it is necessary to wait until the plates are cured. The plates must be used within an hour so that the bacterial growth does not begin before the test samples are placed on them. The test pieces are placed with the active side facing the agar plate and pressed down so that the sample has good contact with the plate, and are incubated in the incubator overnight at 37° C. After that, the zone of inhibition which forms is measured.

H=(D−d)/2

H=inhibition zone [mm]

D=total diameter [mm]

d=diameter of the sample [mm]

Soft Agar Test

In order to obtain more precise results than with the zone of inhibition assay, a defined concentration of bacteria in soft agar is placed on the samples to be tested, and the CFU/mL are assessed after a test period of four hours.

100 mL soft agar are heated to 45° C. and inoculated with 1 mL of the bacteria suspension. Sample pieces measuring 2.5 cm×2.5 cm are punched out with a punch and transferred to empty petri dishes with sterile tweezers. 1 mL of the inoculated soft agar is placed on the surface of the sample pieces with a pipette, care being taken to ensure that the soft agar does not flow down. The latter solidifies at room temperature after approx. 10 minutes. In addition, a positive control is prepared, for this purpose, 1 mL of inoculated soft agar is pipetted into a 50 mL Falcon tube, and 3 mL ¼ strength Ringer's solution is added so that the soft agar does not dry out. The inoculated sample pieces are incubated in the incubator overnight at 37° C. After the test period of four hours, 20 mL Dey-Engley neutralising broth is added in order to bind any free silver ions still present. For the 0-hour value, 1 mL of the inoculated soft agar is pipetted directly into the 20 mL D/E neutralising broth.

The batches are treated for one minute in an ultrasonic bath. After that, suitable dilution series are prepared with ¼ strength Ringer's solution and plated with the pour plate method.

Geometrical mean of the sample=(log x₁+log x₂+log x₃)/(3)

Geometrical mean of the control=(log xk₁+log xk₂+log xk₃)/(3)

Log level reduction=log level control−log level sample

Measuring the Humidity Loss (Dehydration)

The loss of weight over a specific period of time at a defined temperature is described as the humidity loss. The humidity loss is calculated according to the following equation and is stated in the unit g/g:

humidity loss=final weight/initial weight

Measuring the Absorption Capacity

In order to measure the absorption capacity, gel samples with a diameter of 5 cm are punched out. After that, they are placed in a glass beaker with V=300 ml deionised water. Then they are weighed again at certain intervals. The absorption capacity is calculated according to the following equation and is stated in the unit g/g:

absorption capacity=(final weight−initial weight)/initial weight

Test for Cell Compatibility

The tests for cell compatibility were performed in accordance with DIN EN ISO 10993-5 and the methodological instructions of the Department for Functional Materials in Medicine and Dentistry: BioLab 973302, 042901, 964702 and 964805 and comprise measurements of cell growth, metabolic activity and protein content.

The hydrogels were supplied sterile in petri dishes. For the assay, 0.1 g/ml culture medium was weighed into each of the samples.

The cell activity, the cell count and the protein concentration were examined three times per sample in four parallel batches each. The elution time was 48 h. the incubation of the cells with the eluates also 48 h.
Cell line: L 929 CC1 Murine fibroblasts (American Type Culture Collection. Rockeville Md., USA).
Culture medium: DMEM (Dulbecco's mod. Eagle's medium) according to VA BioLab 042901 for the preliminary culture and elution.
Negative control: polystyrene (Nunc GmbH & Co KG, Wiesbaden).
Positive control: Vekoplan KT PVC plates (Konig GmbH, Wendelstein).

Per sample, three eluates from each hydrogel were tested, which were prepared on different test days. For this purpose, the hydrogels were cut through in the middle in the petri dishes with a sterile scalpel and transferred to a sterile 50 ml reaction vessel. Per 0.1 g sample, 1 ml elution medium was added to the hydrogels, and these were then eluted in the incubator for 48 h at 37° C. and 5% CO₂. In order to remove from the eluates any suspended matter present, the samples were centrifuged for 5 min at 4,000 rpm after incubation and filtered through a filter (pore size 0.2 μm).

The cells were seeded in a concentration of 50,000 cells/ml, preculturing was at 37° C. and 5% CO₂ for 24 h. After that, the DMEM medium added during seeding was withdrawn, and the cells were each covered with 1 ml eluate in a concentration of 100%. As a negative control, DMEM medium was incubated in a 50 ml Falcon tube for 48 h like the samples; the eluate from the plastic discs in a concentration of 100% was used as a positive control. After 48 hours of incubation, the cell activity, the cell count and the total protein content were determined.

Cell Growth (Cell Counting)

Cell counting was performed after the enzymatic detachment of the cells by means of Accutase with the aid of the cell counter.

Vitality Test Via Metabolic Activity (WST)

The vitality was tested with tetrazolium salt, WST 1, Roche Diagnostics GmbH Mannheim, in accordance with the manufacturer's instructions. WST 1 is reacted by succinate dehydrogenase (an enzyme of the citric acid cycle) in the mitochondria of the metabolically active cells to yield coloured formazan and measured photometrically. The absorption values(OD), determined at 450 nm and 620 nm, correlate with the breathing activity of the cultured cells.

Protein Content (Lowry)

The protein content was tested with the DC Protein Assay, BIO-RAD GmbH Munich, in accordance with the manufacturer's instructions. The Lowry method of determining protein is based on the reduction of Cu(II) to Cu(I) by the aromatic tyrosine-tryptophan residues of proteins. In a further step, the copper-protein complex reduces a phosphomolybdic acid/phosphotungstate reagent to molybdenum or tungsten blue respectively. The extinction of this intense blue colouring is measured photometrically at 750 nm. The protein concentration can be determined by conducting a standard series at the same time.

Acceptance and Evaluation

The classification of the assessment ranges for acceptance and evaluation was performed in line with DIN EN ISO 7405 and the definition of the inhibition dose (ID 50: dose at which 50% of the cells are inhibited in their growth) (literature: Allgemeine Pharmakologie und Toxikologie, Henschler, ed.: Forth Wolfgang; Spektrum akad. Verl. Heidelberg; 7th ed. 1996). A cell growth of 0-29% is characterised as strong growth inhibition, a cell growth of 30-59% as moderate inhibition and a cell growth of 60-79% as weak inhibition compared to the control. Cell growth rates of between 80 and 100% indicate uninhibited cell growth.

A cell activity of 0-29% is characterised as highly reduced metabolic activity, a cell activity of 30-59% as moderately reduced metabolic activity and a cell activity of 60-79% as slightly reduced metabolic activity compared to the control. Cell activity rates of between 80 and 100% indicate no reduction in metabolic activity.

A protein concentration of 0-34% is characterised as a highly reduced protein content, a protein concentration of 35-69% as a moderately reduced protein content compared to the control. Protein concentrations of between 70 and 100% indicate no reduction in protein content.

The PS value in the presentation of the results corresponds to the polystyrene negative control.

### Example 1: Release of Silver Particles and Silver Ions from the Gel into the Aqueous Medium

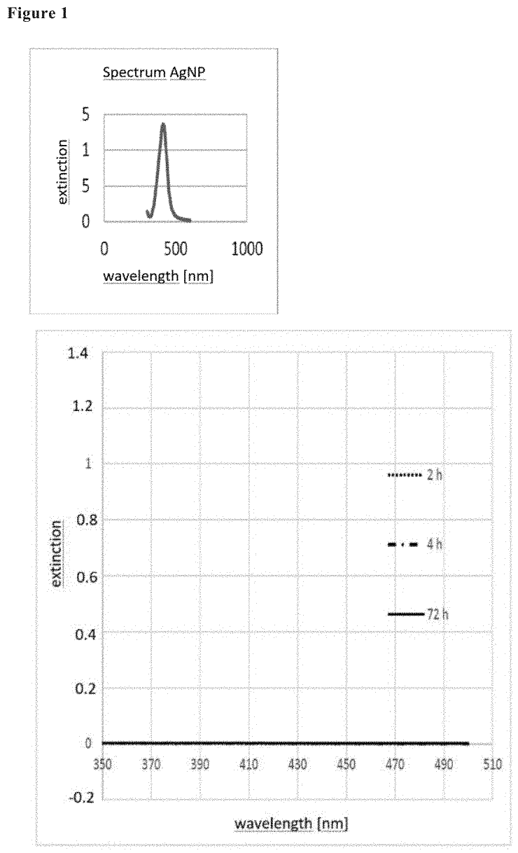

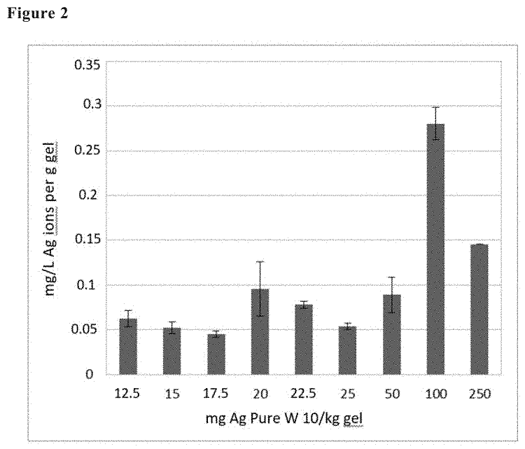

The migration of silver nanoparticles and silver ions from a hydrogel prepared in the manner described above into the aqueous medium was examined by determining the release kinetics. It was established that no detectable quantities of silver particles were released into the aqueous medium (FIG. 1). The quantity of silver ions released was 50 to 300 μg/L Ag+ per 1 g hydrogel, depending on the silver content, as shown in FIG. 2. In simulated wound exudate, a release of silver ions in a range of up to 250 mg/kg hydrogel (250 ppm) was identified.

### Example 2: Antimicrobial Activity of the Hydrogel at Different Silver Contents

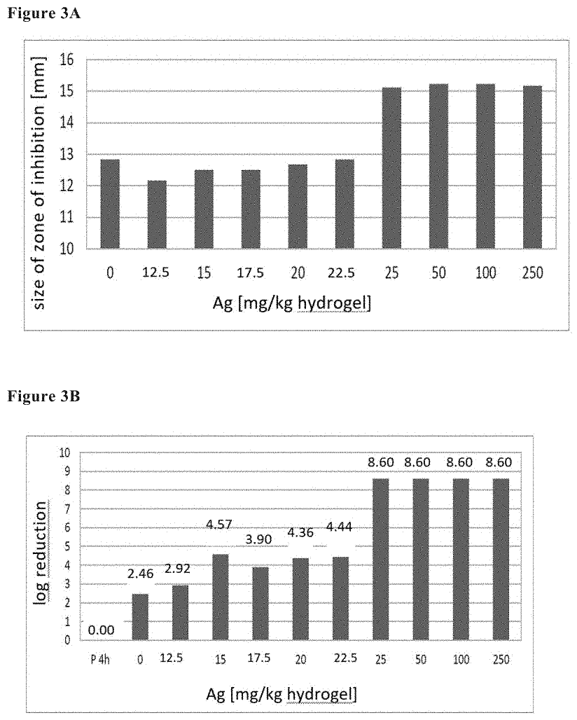

In zone of inhibition assays, a significant inhibition of the growth of Staphylococcus aureus was already observed as of a silver content of 25 mg/kg hydrogel (FIG. 3A. In the range of 25 to 250 mg/kg hydrogel, the inhibition reached 8 log levels (FIG. 3B).

### Example 3: Cytotoxicity and Cell Compatibility of the Hydrogel at Different Silver Contents

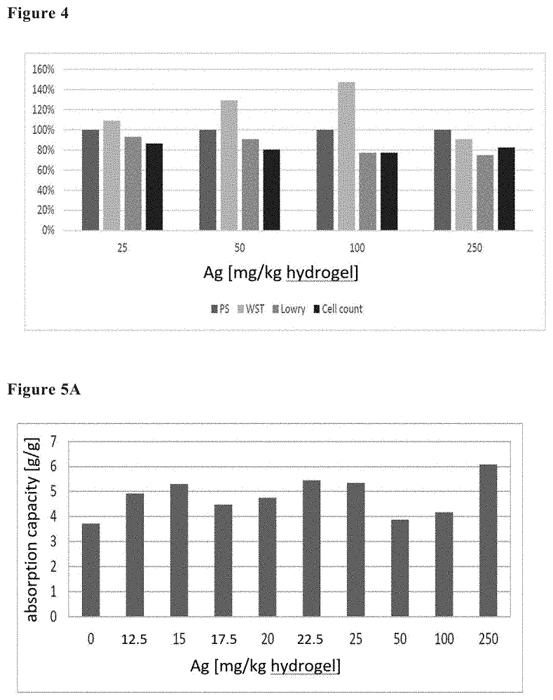

The cytotoxicity and cell compatibility of the hydrogel at different silver contents was established by determining the cell activity, the cell count and the protein concentration, and by means of a vitality test of the metabolic activity (WST). In all the tests, good cell compatibility with low cytotoxicity was established (FIG. 4).

### Example 4: Absorption Capacity of a Hydrogel of the Invention at Different Silver Contents

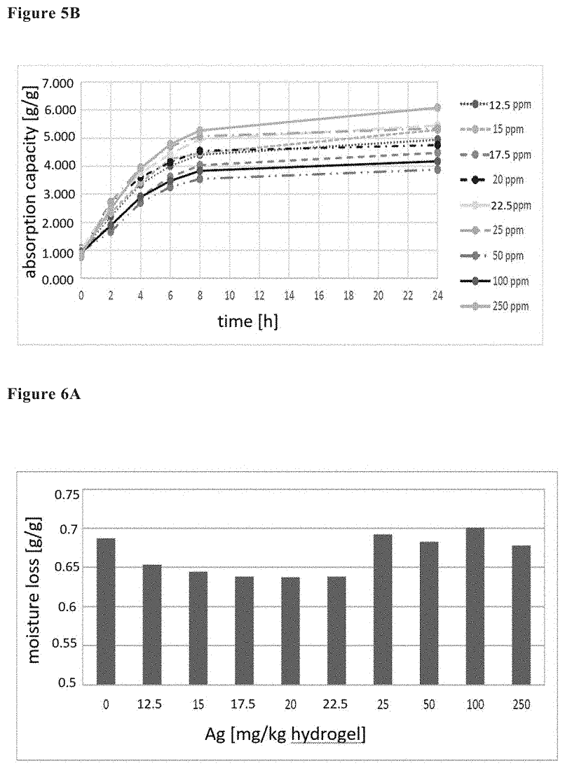

By measuring the absorption capacity of a hydrogel of the invention at different silver contents over a period of up to 24 h, it was not possible to find any change in the absorption capacity depending on the silver content outside any statistical fluctuations (FIGS. 5A and 5B).

### Example 5: Dehydration of a Hydrogel of the Invention at Different Silver Contents

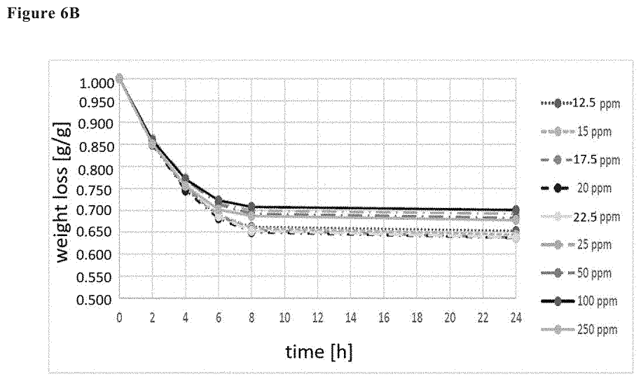

By measuring the dehydration of a hydrogel of the invention at different silver contents over a period of 24 h, it was not possible to find any change in the dehydration depending on the silver content outside any statistical fluctuations (FIGS. 6A and 6B).

It was thus possible by means of the examples shown to demonstrate that by providing a hydrogel composition of the invention, a sufficiently large release of silver ions is possible which ensures effective antimicrobial activity during the treatment of wounds. At the same time, however, the release of silver particles and hence their absorption into the human body is prevented effectively, so that any side-effects caused by this are effectively avoided or can be prevented.

It was also possible to show that the silver particles contained in the hydrogel do not have any influence on its absorption capacity or dehydration, and hence do not impair the function of a wound dressing containing the hydrogel.

By providing the hydrogel composition of the invention, it was thus possible to provide a system for wound therapy with which the treatment of wounds can be performed as effectively as possible and which exhibits high antimicrobial activity. In this way, wound healing can be effectively promoted and accelerated. The wound therapy systems exhibit great wearing comfort and have effective antimicrobial efficacy even when worn for a long time.

1. A water-containing hydrogel composition comprising elemental silver particles with an average particle diameter of 5-20 nm, determined by transmission electron microscopy (TEM), and a particle size distribution of D90≤25 nm, determined by laser diffractometry, wherein the silver content, based on the total weight of the hydrogel composition, is between 15 and 500 ppm.

2. The water-containing hydrogel composition as claimed in claim 1, wherein the average particle diameter is 9-18 nm.

3. The water-containing hydrogel composition as claimed in claim 1, wherein the silver content, based on the total weight of the hydrogel composition, is between 25 and 250 ppm.

4. The water-containing hydrogel composition as claimed in claim 1, wherein the hydrogel composition comprises a hydrophilic polyurethane foam.

5. The water-containing hydrogel composition as claimed in claim 1, wherein the hydrogel composition comprises a polyurethane-polyurea copolymer.

6. The water-containing hydrogel composition as claimed in claim 1, wherein the hydrogel composition further comprises a non-ionic surfactant.

7. The water-containing hydrogel composition as claimed in claim 6, wherein the surfactant is selected from Tween 20, polyethylene glycol, sorbitol and polyvinyl pyrrolidone, or mixtures thereof.

8. The water-containing hydrogel composition as claimed in claim 1, further comprising 5 to 30% by weight, based on the total weight of the hydrogel composition, of a polyhydric alcohol selected from propylene glycol and/or glycerol.

9. The water-containing hydrogel composition as claimed in claim 1, consisting of 6 to 20% by weight of a prepolymer with isophorone diisocyanate ends, 4 to 15% by weight of a diamine based on polyethylene oxide, 15 to 20% by weight propylene glycol and/or glycerol, 40 to 70% by weight water and 25 to 250 ppm elemental silver with an average particle diameter of 9-18 nm, all weight statements based on the total weight of the hydrogel composition.

10. A multi-layered wound dressing, comprising at least one water-impermeable and water vapour-impermeable support layer, an absorbent layer and a layer comprising the water-containing hydrogel composition as claimed in claim 1.

11. The multi-layered wound dressing as claimed in claim 10, wherein the absorbent layer comprises a hydrophilic polyurethane foam and the hydrogel.

12. The multi-layered wound dressing as claimed in claim 11, wherein the surface of the hydrophilic polyurethane foam is at least partially impregnated with the hydrogel.

13. The multi-layered wound dressing as claimed in claim 10, wherein the wound dressing is used in the inflammation phase and/or the granulation phase of wound healing.

14. A method for treating a burn and/or a chronic wound comprising administering the multi-layered wound dressing as claimed in claim 10 to said burn and/or chronic wound.

15. A process for preparing a hydrogel composition, comprising reacting a mixture comprising:
(a) a polyamine,
(b) optionally a non-ionic surfactant,
(c) further optionally a polyhydric alcohol selected from propylene glycol and/or glycerol,
(d) elemental silver particles with an average particle diameter of 5-20 nm, determined by transmission electron microscopy (TEM), and
(e) water,
with an aliphatic diisocyanate polymer in order to form a water-containing hydrogel composition in which the silver content, based on the total weight of the hydrogel composition, is between 15 and 500 ppm.

16. The process for preparing a hydrogel composition as claimed in claim 15, wherein the average particle diameter is 9-18 nm.

17. The process for preparing a hydrogel composition as claimed in claim 15, wherein the silver content, based on the total weight of the hydrogel composition, is between 25 and 250 ppm.

18. The process for preparing a hydrogel composition as claimed in claim 15, wherein the hydrogel composition comprises a hydrophilic polyurethane foam.

19. The process for preparing a hydrogel composition as claimed in claim 15, wherein the hydrogel composition comprises a polyurethane-polyurea copolymer.

20. The process for preparing a hydrogel composition as claimed in claim 15, wherein the hydrogel composition further comprises a non-ionic surfactant.
